(19) United States
(12) Patent Application Publication
(10) Pub. No.: US 2016/0338706 A1
(43) Pub. Date: Nov. 24, 2016

(54) ATRIAL SEPTAL CLOSURE DEVICE FOR RE-ACCESS

(71) Applicant: Edwards Lifesciences Corporation, Irvine, CA (US)

(72) Inventor: Stanton J. Rowe, Newport Coast, CA (US)

(73) Assignee: Edwards Lifesciences Corporation, Irvine, CA (US)

(21) Appl. No.: 15/159,012

(22) Filed: May 19, 2016

Related U.S. Application Data
(60) Provisional application No. 62/164,392, filed on May 20, 2015.

(51) Int. Cl. A61B 17/12

(52) U.S. Cl. CPC A61B 17/1214; A61B 17/12022; A61B 2017/1205

(57) ABSTRACT

In particular embodiments, a septal orifice closure device for closing a septal defect can include a frame structure comprising a coil having a first loop turn, a second loop turn and a third loop turn. A biodegradable member can be attached to the second loop turn, for example. The second loop turn can be sandwiched between the first and second loop turns. The biodegradable member can be replaced by the body with scar tissue formation and endothelial cells such that only the frame member remains in the body after a period of time. The lumen can be configured to allow a medical device to be inserted through the device at the location of the previous orifice at a later time as other therapeutic interventions are warranted.

### CROSS REFERENCE TO RELATED APPLICATION

This application claims the benefit of U.S. Provisional Patent Application No. 62/164,392, filed May 20, 2015, which is incorporated herein by reference.

### FIELD

The present disclosure relates generally to a method and device for closing a septal orifice, or opening in the septum of a heart. In particular, the present disclosure relates to a method and device for closing an orifice in an atrial septum, such that the septal orifice can be accessed for reentry at the location of the orifice.

### BACKGROUND

A septum may include a thin wall dividing a cavity into two smaller structures. An atrial septum is a wall of tissue separating the left and right atrial of the heart. A ventricular septum is a wall of tissue separating the left and right ventricles of the heart. A septal defect or orifice may include a perforation or hole passing through the septum. A septal defect can occur congenitally or by puncturing the septum with a medical device to access a location within the heart.

The atrial septum may be viewed like the femoral artery in years to come. The femoral artery is an access point for many catheterization procedures, with a smaller percentage of procedures utilizing venous or radial artery access Likewise, the atrial septum is a point of percutaneous access for atrial fibrillation therapy, left atrial appendage closure, percutaneous mitral valve repair, and percutaneous mitral valve replacement. In these and other procedures, devices may traverse across the atrial septum and, by doing so, may leave a defect in the atrial septum that cannot close spontaneously. Currently, these defects are closed using devices, such as plugs, that may close the defect but do not allow for re-access through the septum. Thus, a need exists for improved closure devices for closing a septal defect and optionally for re-accessing the left side of the heart in subsequent procedures.

### SUMMARY

The method and device of the present disclosure may be used to close a septal orifice, such that the septum can be re-accessed at a later time as other therapeutic interventions are warranted. The septal orifice may be any opening in a septum including a congenital defect, an iatrogenic defect, and/or an opening formed in the septum to perform a medical procedure.

The device can include a frame structure having one or more segments. The one or more segments can include a distal anchor configured for placement in a left atrium and a proximal anchor for placement in the right atrium. The profile of the distal and proximal anchors can be minimalistic so as to limit the risk of thrombus formation in the left and right atriums, which may cause stroke. The one or more segments can include a sealing member or flow-blocking member comprising a biodegradable membrane or biological sheet configured to extend over and occlude the septal orifice and allow for native ingrowth of tissue. The proximal anchor can be further configured to hold or sandwich the biodegradable sealing member against the right atrial septal wall. Alternatively, the distal anchor can be configured to hold or sandwich the biodegradable sealing member against the left atrial septal wall.

The sealing member can be configured to degrade or dissolve in the body over time, desirably after a time period sufficient to allow the septal orifice to heal. If a subsequent trans-septal procedure is required, such as to repair or replace tissue in the left side of the heart, the left side of the heart can be accessed by inserting a catheter through the frame structure of the closure device and through the septum at the same location of the previous septal orifice.

In one representative embodiment, a septal orifice closure device comprises a helical frame comprising at least two turns and defining a central lumen, and at least one biodegradable member attached to the frame and configured to block the flow of blood through the lumen and between the left and right atriums when the frame is deployed within a septal orifice in a septum of a heart of a patient.

In some embodiments, the coil frame comprises at least three turns. In some embodiments, the at least three turns comprise a first turn, a second turn, and a third turn, the second turn being intermediate the first and third turns, and the first and third turns having respective diameters that are greater than a diameter of the second turn. In some embodiments, the at least three turns comprise a first turn, a second turn, and a third turn, the second turn being intermediate the first and third turns, and the first and third turns having respective diameters that are less than a diameter of the second turn.

In some embodiments, the biodegradable member is sutured to the frame.

In some embodiments, the frame comprises a coiled wire.

In some embodiments, the frame is configured such that adjacent turns of the frame are biased against each other in an expanded configuration.

In some embodiments, the at least one biodegradable member comprises first and second biodegradable members attached to the frame which are configured to be positioned on opposite sides of the septum when the frame is deployed in the septal orifice

In another representative embodiment, a septal orifice closure device comprises a frame comprising a distal anchor and a proximal anchor, the distal anchor configured for placement on a first side of a septal orifice in a septum of a heart, the proximal anchor configured for placement on a second side of the septal orifice. The closure device can further comprise at least one biodegradable sealing segment supported on the frame and configured to block the flow of blood through the septal orifice when the distal anchor is deployed on the first side of the septal orifice and the proximal anchor is deployed on the second side of the septal orifice.

In some embodiments, the frame comprises a helical frame comprising two or more turns, wherein the distal and proximal anchors each comprise one of the turns of the frame.

In some embodiments, the frame is configured to clamp the septum between adjacent turns of the anchor.

In some embodiments, the at least one sealing segment comprises a first sealing segment supported on the distal anchor and a second sealing segment supported on the proximal anchor.

In some embodiments, the at least one biodegradable sealing segment comprises a circular piece of material secured along its circumferential edge to the frame.

In another representative embodiment, a medical procedure comprises inserting a delivery catheter into the vasculature of a patient, the delivery catheter comprising a sheath containing a septal closure device in a compressed configuration, the closure device comprising a frame and at least one biodegradable flow-blocking member supported on the frame. The method further comprises advancing at least a distal end portion of the sheath through an orifice in the atrial septum of the patient's heart, and deploying the closure device from the sheath such that a first portion of the frame is deployed in the left atrium, a second portion of the frame is deployed in the right atrium, and the flow-blocking member blocks the flow of blood through the orifice.

In some embodiments, the method further comprises, after the flow-blocking member dissolves in the body and the orifice is occluded by tissue growth, inserting a medical instrument through the frame and the septum at the prior location of the orifice and performing a medical procedure in the left side of the heart using the medical instrument. In some embodiments, the medical instrument comprises a delivery catheter and a prosthetic heart valve carried on a distal end portion of the delivery catheter, and performing a medical procedure comprises implanting the prosthetic heart valve in the native mitral valve annulus of the heart.

In some embodiments, the frame comprises a helical anchor comprising two or more turns.

In some embodiments, the at least one biodegradable flow-blocking member comprises first and second biodegradable flow-blocking members and the act of deploying the closure device comprises deploying the first and second biodegradable flow-blocking members on opposite sides of the atrial septum.

In some embodiments, the sheath retains the frame in an uncoiled configuration during the act of inserting the delivery catheter into the vasculature of the patient and the frame self-expands to a coiled configuration as it is deployed from the sheath.

In some embodiments, a portion of the atrial septum surrounding the orifice is compressed between the first and second portions of the frame when the closure device is deployed.

The foregoing and other objects, features, and advantages of the invention will become more apparent from the following detailed description, which proceeds with reference to the accompanying figures.

### BRIEF DESCRIPTION OF THE DRAWINGS

FIG. 1 is a cross-sectional view of a heart with an atrial septal defect.

FIG. 2 is a perspective view of an embodiment of a closure device in accordance with on embodiment of the present disclosure.

FIG. 3A-3B are perspective and elevation views, respectively, of a closure device in accordance with the present disclosure.

FIG. 4 is a perspective view of an embodiment of a first segment of a closure device deployed adjacent a septum in accordance with the present disclosure.

FIG. 5 is an elevation view of an embodiment of a second segment of a closure device in accordance with the present disclosure.

FIG. 6 is a perspective view of an embodiment of a third segment of a closure device deployed adjacent a septum in accordance with the present disclosure.

FIG. 7 is a side view of an embodiment of a closure device after having been implanted in the atrial septum.

FIG. 8 is a side view of a distal end portion of a delivery apparatus and a closure device retained in the delivery apparatus for delivery into a patient, according to one embodiment.

FIG. 9 is a perspective view of an alternative embodiment of a closure device in accordance with the present disclosure.

FIG. 10 is a perspective view of an alternative embodiment of a closure device in accordance with the present disclosure.

FIG. 11 is a perspective view of an alternative embodiment of a closure device in accordance with the present disclosure.

FIG. 12 is an elevation view of an alternative embodiment of a closure device in accordance with the present disclosure.

### DETAILED DESCRIPTION

For purposes of this description, certain aspects, advantages, and novel features of the embodiments of this disclosure are described herein. Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention extends to any novel one, or any novel combination, of the features disclosed in this specification (including any accompanying claims, abstract and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an”, and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.”

As used herein, the term “coupled” generally means physically coupled or linked and does not exclude the presence of intermediate elements between the coupled items absent specific contrary language.

The present disclosure describes a closure device that is suitable to close or repair a septal orifice and allow for re-entry through the septum at the same location at a later time as other therapeutic interventions are warranted. For example, the closure device can allow a catheter or other medical device to be inserted through the closure device to access the left side of the heart in a subsequent procedure. As used herein, the term “septal orifice” or “orifice” is used to describe an orifice created by puncturing the septum with a catheter or other medical device and an orifice that occurs congenitally, such as an atrial septal defect (ASD) or a patent foramen ovale (PFO).

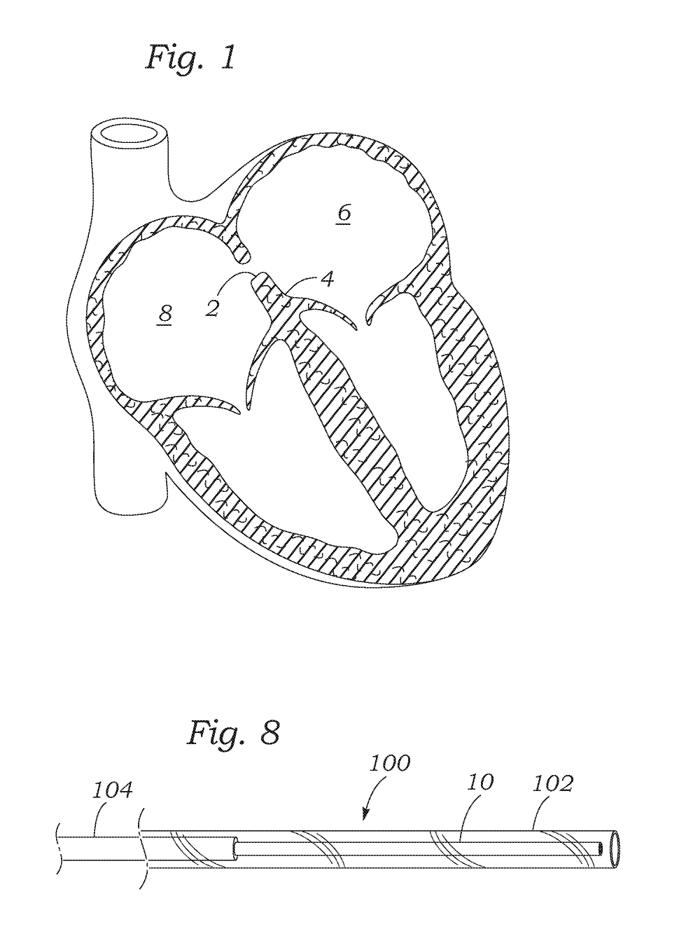

FIG. 1 illustrates a heart shown in cross section having a septal orifice 2 in an atrial septum 4 between a left atrium 6 and a right atrium 8. The septal orifice 2 may be created, for example, by a catheter or other medical instrument that is pushed through the atrial septum 4 to repair or replace a valve or other tissue in the left side of the heart. The closure device described herein is suitable to repair the septal orifice 2, such that the atrial septum 4 is sealed, and allow for re-entry through the atrial septum 4 at the same location of the septal orifice 2 at a later time. For continuity, certain reference numbers and symbols will be used throughout this disclosure, though multiple embodiments will be disclosed.

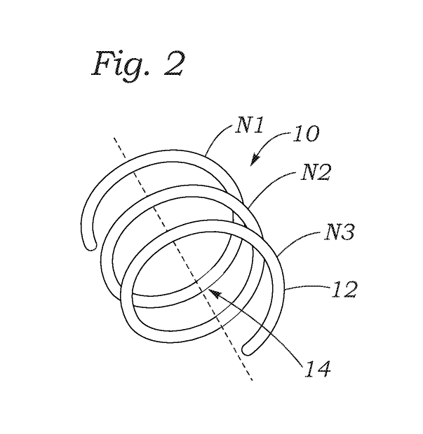

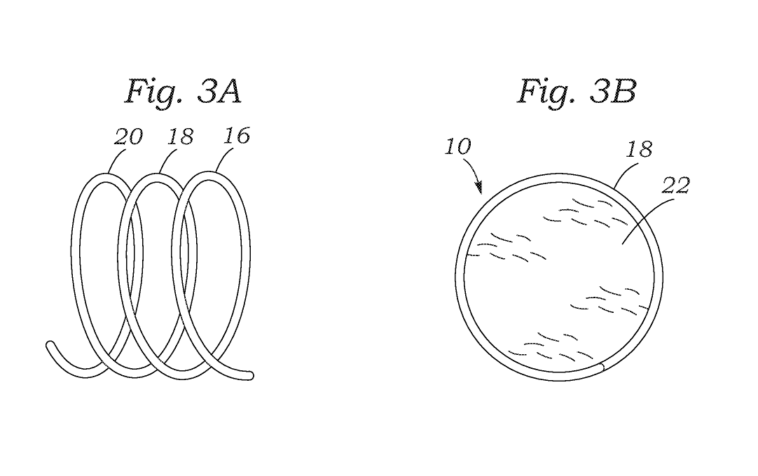

An exemplary closure device 10 is illustrated in FIG. 2. The closure device 10 can include a frame structure 12 configured to support a sealing member 22 (also referred to as a “flow-blocking member”) (shown in FIG. 3B) comprising a biodegradable membrane or a biological sheet (e.g., a biodegradable polymer membrane), also referred to as a biodegradable member, that allows for native ingrowth of tissue as the biodegradable sheet degrades in the body. The biodegradable sheet initially forms a barrier to blood flow through the defect. Over time, the biodegradable sheet is replaced by growth of scar tissue formation and endothelial cells. The frame structure 12 desirably is left coated with the body's own material, which blocks the septal defect.

The frame structure 12 material(s) can have shape memory such that when the material(s) are deformed from a preformed, set shape, the material(s) will naturally reform into the original shape when no longer deformed. The frame structure 12 material can be formed from a shape memory metal such as Nitinol wire or any other appropriate wire or other material. The frame structure 12 can comprise a continuous structure of a single material or can include two or more sections of different material connected to each other (e.g., by welding) and/or sections having different preformed, heat-set shapes.

The frame structure 12 can further include at least one core area or lumen 14. The lumen 14 can be configured such that it is substantially open and no portion of the frame structure 12 extends into or impedes the path of a device inserted into the lumen 14. In particular embodiments, the frame structure 12 comprises a helical shape having N number of turns, loops, rings, or coils. The lumen 14 can extend unobstructed through the N number of turns. In the embodiment illustrated in FIG. 2, the closure device 10 includes three turns, N1-N3, although the closure device can have a greater or fewer number of turns N. For example, in some embodiments, the frame 12 can comprises a helical structure having two turns or four or more turns. Each turn can have the same size or can be sized differently than the others. At least one turn can be sized larger than the septal orifice in which the closure device is implanted. For example, the frame 12 can comprise a helical structure having a relatively larger center turn that is larger than the septal orifice and end turns on opposite sides of the center turn that can be smaller in diameter than the center turn. The lumen 14 can have the same diameter as the smallest turn of the coil frame. The turns can have any appropriate shape, including round, oval, square, helical, or combinations thereof.

FIG. 3A-3B are different views of an embodiment of a closure device 10 having a first segment 16, a second segment 18 and a third segment 20. Each segment can be the same size or can be sized differently. The first, second and third segments 16, 18, 20 can be constructed of a single continuous wire or tubular structure, or alternatively, three wires or tubular structures connected end-to-end. A single wire device can provide simplicity of deployment and a low profile afforded a device which can be elongate in a collapsed state. An exemplary material can be Nitinol wire, however other appropriate materials can be used. The first, second and third segments 16, 18, 20 can be substantially or totally ring shaped. One or more of the first, second or third segments 16, 18, 20 can form an open ring, i.e., a ring extending less than 360 degrees around its circumference, or a closed ring, i.e., a ring extending 360 degrees or more around its circumference.

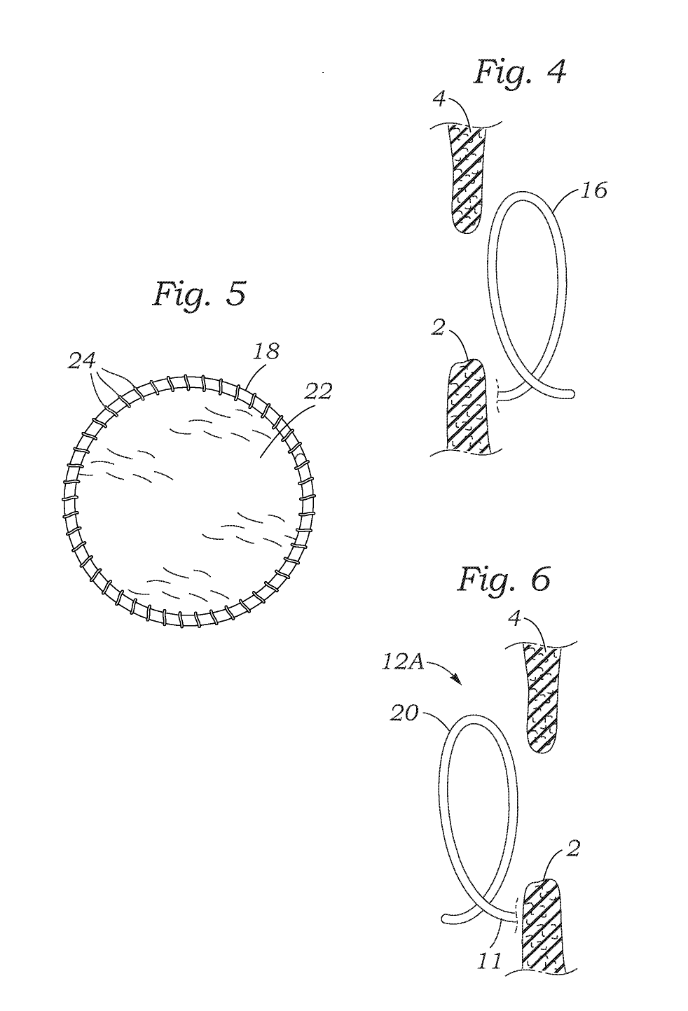

As showing in FIG. 3A, the first segment 16, which can be referred to as a distal anchor in the illustrated embodiment, can be configured for placement in the left atrium. The profile of the first segment 16 can be minimalistic, so as to limit the risk of thrombus formation in the left atrium, which may cause stroke. As shown in FIG. 4, the first segment 16 can be biased towards the atrial septum 4 to secure other segments of the closure device 10, such as the second segment 18, against the septum.

As shown in FIG. 3B, the second segment 18, which can be referred to as the membrane section or sealing segment in the illustrated embodiment, can include at least a frame portion having at least a partial loop configuration and a biodegradable membrane or biological sheet 22 disposed across the partial loop configuration. Exemplary biodegradable membrane materials can include one or more of any of various suitable natural tissues (e.g., non-glutaraldehyde fixed pericardium or porcine SMS) or polymeric materials (e.g., polyglycolic acid (PGA), polylactic acid (PLA) or PGA/PLA copolymers). The biological sheet 22 can be fastened to the frame by any appropriate means including biodegradable sutures 24 (see FIG. 5), heat bonding or appropriate adhesive. The diameter of the biological sheet 22 can be the same as diameter of the frame. The diameter of the frame, at least at the second segment 18, can be larger than the septal orifice 2.

The third segment 20, which can be referred to as the proximal anchor in the illustrated embodiment, can be configured for placement in the right atrium. As shown in FIG. 6, the third segment 20 can be biased towards the septum 4 to secure other segments of the closure device 10, such as the second segment 18, against the septum.

In some embodiments, the entire closure device 10, including the frame 12, can be formed from a biodegradable material that dissolves in the body after a certain time.

The closure device 10 can be implanted percutaneously using a delivery apparatus 100, shown in FIG. 8. The delivery apparatus 100 can include an outer catheter or sheath 102 and an inner shaft or pusher rod 104. The closure device 10 can initially be in a deformed, substantially straight or extended form such that it lies within the lumen of outer sheath 102.

The delivery apparatus 100 can be advanced percutaneously through the patient's vasculature to the right atrium 8 of the heart in a trans-septal, antegrade approach for implanting the closure device 10 in the septum 4. In one approach, the delivery apparatus 100 can be advanced through a femoral vein, the inferior vena cava, and into the right atrium. In another approach, the delivery apparatus can be advanced through a vein of the upper torso (e.g., a jugular vein), the superior vena cava, and into the right atrium.

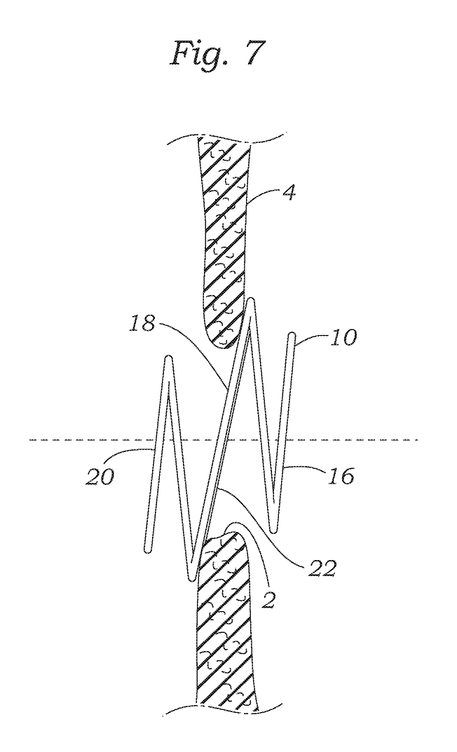

Once inside the right atrium, the delivery apparatus 100 can be advanced through the septal defect 2 such that the distal end portion of the delivery sheath 102 extends into the left atrium 6. The closure device 10 can then be deployed from the distal tip of the outer sheath 102 by pushing the inner shaft 104 distally and/or retracting the outer sheath 102 proximally, such that the first segment 16 is deployed in the left atrium and returns to its coiled, shape-set state (such as shown in FIG. 4). The second segment 18 of the closure device 10 can be at least partially deployed within the left atrium 6 (such as shown in FIG. 7). The delivery apparatus 100 then can be retracted slightly back through the septal defect such that the deployed portion of the second segment 18 abuts the septum and the distal tip of the delivery sheath 102 is within the right atrium.

The remainder of the closure device 10 can be deployed from the tip of the outer sheath 102 by pushing the inner shaft 104 distally and/or retracting the outer sheath 102 proximally, such that the remainder of the second segment 18 and the third segment 20 are deployed in the right atrium 8 and return to their coiled, shape-set state. As shown in FIG. 7, a portion of the second segment 18 can lie against the septum in the left atrium and another portion of the second segment 18 can lie against the septum in the right atrium such that the biodegradable sheet 22 spans across and covers the septal defect 2.

FIG. 7 shows there is spacing between adjacent segments or turns 16, 18, 20 of the coil frame 12 in its expanded state. In other embodiments, the coil frame 12 can be configured such that there is no spacing between the individual segments 16, 18, 20 (the segments are biased against each other) when the closure device is in its expanded state. When deployed in the septum, the first and third segments 16, 20 can contact opposite sides of the second segment 18 and/or the septum 4. In this manner, the first and third segments 16, 20 can assist in anchoring the closure device 10 in place by exerting a clamping force against opposite sides of the septum 4. In some implementations, each segment 16, 18, 20 can have the same diameter, and the first and third segments 16, 20 can abut opposite sides of the second segment 18. In other implementations, the first and third segments 16, 20 can have diameters larger or smaller than the diameter of the second segment 18, and the first and third segments can contact opposite sides of the septum 4 when fully deployed.

In alternative embodiments, the closure device 10 can be implanted such that the second segment 18 can lie totally in the left atrium 6 or totally in the right atrium 8. For example, the closure device 10 can be deployed such that the septum 4 is positioned or clamped between the first segment 16 and the second segment 18, or alternatively, between the second segment 18 and the third segment 20.

In some embodiments, the three segments 16, 18, 20 can include interlocks keyed for orientation. At each transition point, from one segment to the next, the delivery apparatus 100 can include stops or marks (e.g., radiopaque markings) that instruct the cardiologist to orient the closure device 10 into its optimal position. Additionally, in some embodiments, the frame 12 can include radiopaque markings along its length, for example, at the transition points between adjacent segments to assist the user in deploying each segment in its desired location in the heart.

In alternative embodiments of the closure device 10, one or more of each segment 16, 18, 20 of the frame 12 can include a respective biodegradable sheet 22. In some implementations, for example, a biodegradable sheet 22 can be supported on more than one segment of the closure device such that when the closure device is implanted, at least one biodegradable sheet is positioned adjacent the septal orifice in the left atrium and at least one biodegradable sheet is positioned adjacent the septal orifice in the right atrium.

After the biodegradable sheet 22 has degraded and the septal orifice 2 has closed, the same location on the septum can be used to access the left atrium with a catheter or other medical instrument in a subsequent procedure. A catheter, for example, can be pushed through the lumen 14 of the frame structure 12 and can create an opening at the location of the previous septal orifice.

The catheter used in a subsequent procedure can be, for example, a delivery apparatus for delivering and implanting a prosthetic heart valve in the native mitral valve or the native aortic valve. In alternative embodiments, the delivery apparatus can be used to deliver and implant various other prosthetic devices in the left atrium, mitral valve, left ventricle, and/or the aortic valve, including, for example, annuloplasty rings, closure devices for the left atrial appendage, sealing devices or reshaping devices for repairing or reshaping portions of the heart. In other embodiments, other percutaneous medical instruments can be advanced through the lumen 14 of the frame structure 12 for performing a procedure on the left side of the heart, such as atrial fibrillation therapy.

If the medical instrument has a relatively small diameter, such as used for treating arrhythmias, the hole formed in the septum can be small enough to sufficiently inhibit blood flow between the left and right atriums without further intervention. If the medical instrument has a relatively large diameter, such as a delivery apparatus for implanting prosthetic valve, and leaves a relatively larger opening in the septum, another closure device 10 can be implanted within the remaining frame 12 of the first closure device to block blood flow between the right and left atriums.

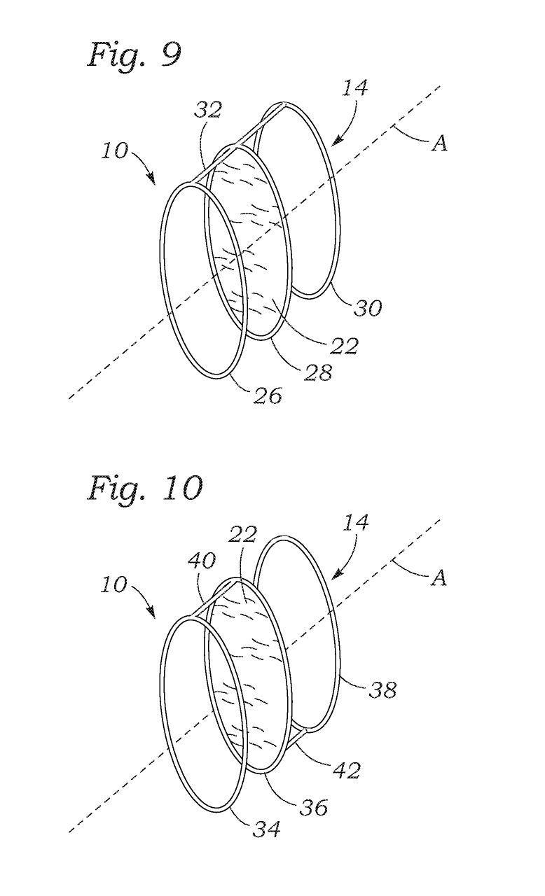

FIGS. 9-11 illustrate alternative embodiments of the closure device 10. FIG. 9 shows a closure device 10 comprising a frame 25. The frame 25 comprises a plurality of complete loops or rings 26, 28, 30 connected to each other by a single connecting bar 32 that is attached at the same location on each loop 26, 28, 30. The multiple loops 26, 28, 30 and connecting bar 32 can be constructed from a continuous wire or can be constructed of multiple segments of wire that are welded or otherwise fixed together. The connecting bar 32 can lie on an axis parallel to a central axis A of the lumen 14 of the device. One or more of the multiple loops 26, 28, 30 can include a biodegradable sheet 22 spanning the lumen 14. In an alternative embodiment, the frame 25 can comprise only two loops 26, 28.

FIG. 10 shows a closure device 10 comprising a frame 33. The frame 33 comprises a plurality of complete loops or rings 34, 36, 38, a first connecting bar 40 and a second connecting bar 42. The first connecting bar 40 connects two loops 34, 36 at a first location on each loop 34, 36. The second connecting bar 42 connects two loops 36, 38 at a second location on each loop 36, 38. For example, the first connecting bar 40 and the second connecting bar 42 can be located at diametrically opposing locations from each other with respect to a central axis A of the lumen 14 of the device 10. The first and second connecting bars 40, 42 can lie along respective axes that are parallel to the central axis A. One or more of the multiple loops can include a biodegradable sheet 22 spanning the lumen 14.

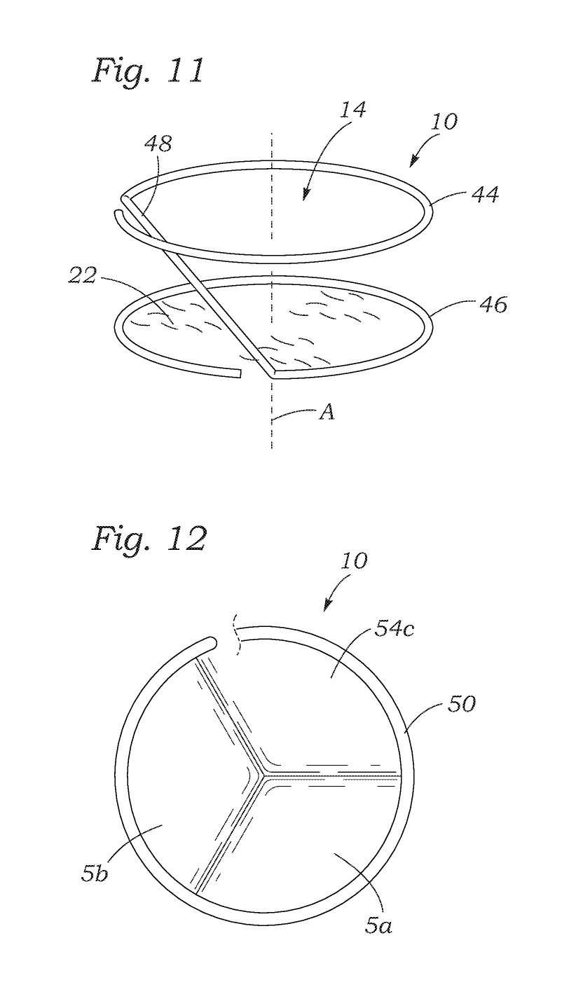

FIG. 11 shows a closure device 10 comprising a first loop 44 and a second loop 46, which may be partial loops (as shown) or complete loops (loops that circumscribe a central axis through 360 degrees). The first and second loops 44, 46 can be connected by a connecting bar 48. As shown, the connecting bar 48 can be skewed with respect to a central axis A of the device. A biodegradable sheet 22 can be attached to the second loop 46 (as shown in FIG. 11) and/or to the first loop 44.

In alternative embodiments, a closure device 10 can comprise a sheet 22 of material that is substantially non-degradable within the body. In such embodiments, the sheet 22 can be formed from any of various suitable materials, including natural tissue or synthetic materials, such as any of various woven (e.g., fabric) or non-woven materials made from any of various polymeric materials. Some examples of natural tissue include, for example, bovine, porcine, or equine pericardial tissue or pericardial tissue from other animals. Some examples of suitable polymeric materials include, for example, polyurethane or polyester. A suitable fabric material includes, for example, polyethylene terephthalate (PET) fabric. In some implementations, the sheet 22 is selected to permit a medical instrument (e.g., a delivery catheter) to puncture the sheet 22 and cross the atrial septum in a subsequent procedure. For example, the sheet 22 can be a thin piece of tissue (e.g., pericardial tissue) or a thin polymeric sheet that can be punctured by the distal end portion of a medical instrument.

In some implementations, a closure device can comprise a valve member instead of a unitary sheet 22 that blocks the flow of blood through the device. The valve member can be configured to block the flow of blood between the left atrium and the right atrium but permit a medical instrument to be easily inserted through the closure device to access the left side of the heart. FIG. 12, for example, shows a closure device 10 comprising a segment 50 supporting a valve member 52. Although a single frame segment 50 is shown for purposes of illustration, the closure device 10 can include the frame of any of the closure devices described herein. For example, the segment 50 can be one of the first, second, or third segments 16, 18, 20 of the frame 12 described above.

The valve member 52 can comprise a plurality of flaps or leaflets 54a, 54b, 54c. The flaps can be biodegradable or non-biodegradable and can be formed from any of the biodegradable and non-biodegradable materials described above for forming the sheet 22. In some embodiments, the flaps can be configured to block the flow of blood between the left and right atriums, but allow a medical instrument to be inserted through the flaps to access the left side of the heart during a subsequent procedure.

In view of the many possible embodiments to which the principles of the disclosed invention can be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of these claims.

I claim:

1. A septal orifice closure device comprising:
a helical frame comprising at least two turns and defining a central lumen; and
at least one biodegradable member attached to the frame and configured to block the flow of blood through the lumen and between the left and right atriums when the frame is deployed within a septal orifice in a septum of a heart of a patient.

2. The closure device of claim 1, wherein the coil frame comprises at least three turns.

3. The closure device of claim 2, wherein the at least three turns comprise a first turn, a second turn, and a third turn, the second turn being intermediate the first and third turns, and the first and third turns having respective diameters that are greater than a diameter of the second turn.

4. The closure device of claim 2, wherein the at least three turns comprise a first turn, a second turn, and a third turn, the second turn being intermediate the first and third turns, and the first and third turns having respective diameters that are less than a diameter of the second turn.

5. The closure device of claim 1, wherein the biodegradable member is sutured to the frame.

6. The closure device of claim 1, wherein the frame comprises a coiled wire.

7. The closure device of claim 1, wherein the frame is configured such that adjacent turns of the frame are biased against each other in an expanded configuration.

8. The closure device of claim 1, wherein the at least one biodegradable member comprises first and second biodegradable members attached to the frame which are configured to be positioned on opposite sides of the septum when the frame is deployed in the septal orifice

9. A septal orifice closure device comprising:
a frame comprising a distal anchor and a proximal anchor, the distal anchor configured for placement on a first side of a septal orifice in a septum of a heart, the proximal anchor configured for placement on a second side of the septal orifice; and
at least one biodegradable sealing segment supported on the frame and configured to block the flow of blood through the septal orifice when the distal anchor is deployed on the first side of the septal orifice and the proximal anchor is deployed on the second side of the septal orifice.

10. The closure device of claim 9, wherein the frame comprises a helical frame comprising two or more turns, wherein the distal and proximal anchors each comprise one of the turns of the frame.

11. The closure device of claim 10, wherein the frame is configured to clamp the septum between adjacent turns of the anchor.

12. The closure device of claim 9, wherein the at least one sealing segment comprises a first sealing segment supported on the distal anchor and a second sealing segment supported on the proximal anchor.

13. The closure device of claim 9, wherein the at least one biodegradable sealing segment comprises a circular piece of material secured along its circumferential edge to the frame.

14. A medical procedure comprising:
inserting a delivery catheter into the vasculature of a patient, the delivery catheter comprising a sheath containing a septal closure device in a compressed configuration, the closure device comprising a frame and at least one biodegradable flow-blocking member supported on the frame;
advancing at least a distal end portion of the sheath through an orifice in the atrial septum of the patient's heart; and
deploying the closure device from the sheath such that a first portion of the frame is deployed in the left atrium, a second portion of the frame is deployed in the right atrium, and the flow-blocking member blocks the flow of blood through the orifice.

15. The method of claim 14, further comprising, after the flow-blocking member dissolves in the body and the orifice is occluded by tissue growth, inserting a medical instrument through the frame and the septum at the prior location of the orifice and performing a medical procedure in the left side of the heart using the medical instrument.

16. The method of claim 14, wherein the medical instrument comprises a delivery catheter and a prosthetic heart valve carried on a distal end portion of the delivery catheter, and performing a medical procedure comprises implanting the prosthetic heart valve in the native mitral valve annulus of the heart.

17. The method of claim 14, wherein the frame comprises a helical anchor comprising two or more turns.

18. The method of claim 14, wherein at least one biodegradable flow-blocking member comprises first and second biodegradable flow-blocking members and the act of deploying the closure device comprises deploying the first and second biodegradable flow-blocking members on opposite sides of the atrial septum.

19. The method of claim 14, wherein the sheath retains the frame in an uncoiled configuration during the act of inserting the delivery catheter into the vasculature of the patient and the frame self-expands to a coiled configuration as it is deployed from the sheath.

20. The method of claim 19, wherein a portion of the atrial septum surrounding the orifice is compressed between the first and second portions of the frame when the closure device is deployed.
